(19) United States
(12) Patent Application Publication
(10) Pub. No.: US 2019/0196664 A1
(43) Pub. Date: Jun. 27, 2019

(54) MEDICAL IMAGE DISPLAY SYSTEM AND MEDICAL IMAGE DISPLAY APPARATUS

(71) Applicant: Konica Minolta Inc., Tokyo (JP)

(72) Inventor: Naotoshi SUGEGAYA, Tokyo (JP)

(21) Appl. No.: 16/227,631

(22) Filed: Dec. 20, 2018

(30) Foreign Application Priority Data
Dec. 25, 2017 (JP) 2017-247215

(51) Int. Cl. G06F 3/0481; G06F 3/0488; G06F 3/0484; A61B 90/00

(52) U.S. Cl. CPC G06F 3/0481; G06F 3/04883; G06T 11/60; A61B 90/37; G06F 2203/04803; G06F 3/0484

(57) ABSTRACT

A medical image display system includes a medical image display apparatus that displays a medical image on a display screen of a touch panel, the medical image display system being capable of changing a display mode of the medical image displayed on the display screen on the basis of screen contact operation on the display screen, wherein the display screen includes: an image display region that displays the medical image; and a small region being a region smaller than the image display region and arranged at a position where the medical image is not displayed, and a display mode of the image display region is set so as to change on the basis of the screen contact operation in the small region.

### CROSS REFERENCE TO RELATED APPLICATION

The present application claims priority under 35 U.S.C. § 119 to Japanese patent Application No. 2017-247215, filed on Dec. 25, 2017, is incorporated herein by reference in its entirety.

### BACKGROUND

### Technological Field

The present invention relates to a medical image display system and a medical image display apparatus.

### Description of the Related Art

There is a known medical image display apparatus capable of changing a display mode of a medical image displayed on a touch panel on the basis of an instruction on a touch panel such as a smartphone or a tablet terminal.

JP 2016-123584 A discloses a technique capable of performing a simulation of first defining a first point and a second point designated by an operator on a touch panel of a display terminal and then dividing an anatomical structure as a subject into a plurality of parts by using a line defined by the first and second points as a cutting reference line.

JP 2017-074213 A discloses a technique used at the time of diagnosis performed by displaying an ultrasound image on a touch panel of a display terminal, in which a user such as a doctor or an laboratory technician performs touch operation by a finger or the like on a display screen of the touch panel using touch points of the number of thresholds or more so as to perform freeze control of the ultrasound image.

The techniques disclosed in JP 2016-123584 A and JP 2017-074213 A, however, includes a problem of degradation of visibility due to a state where the view of the medical image is hidden by a finger or a hand because of operation on the medical image displayed on the touch panel. In addition, since operation uses almost a whole region on the touch panel, it is not easy to operate with a single hand with a relatively large terminal such as a tablet terminal.

### SUMMARY

An object of the present invention is to achieve suppression of degradation of visibility of a medical image displayed on a display screen and as well as enhancing operability.

To achieve the abovementioned object, according to an aspect of the present invention, a medical image display system reflecting one aspect of the present invention comprises: a medical image display apparatus that displays a medical image on a display screen of a touch panel, the medical image display system being capable of changing a display mode of the medical image displayed on the display screen on the basis of screen contact operation on the display screen, wherein the display screen includes: an image display region that displays the medical image; and a small region being a region smaller than the image display region and arranged at a position where the medical image is not displayed, and a display mode of the image display region is set so as to change on the basis of the screen contact operation in the small region.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a block diagram illustrating a configuration of a medical image display apparatus;

FIG. 2 is a diagram illustrating a display example of a display screen;

FIG. 3 is a diagram illustrating a display example of a display screen;

FIG. 4 is a diagram illustrating a display example of a display screen;

FIG. 5 is a diagram illustrating a display example of a display screen;

FIG. 6 is a diagram illustrating a display example of a display screen.

FIG. 7 is a diagram illustrating a display example of a display screen;

FIG. 8 is a diagram illustrating a display example of a gesture registration screen;

FIG. 9 is a diagram illustrating a display example of a gesture registration screen;

FIG. 10 is a diagram illustrating a display example of a display screen;

FIG. 11 is a diagram illustrating a display example of a display screen;

FIG. 12 is a diagram illustrating a display example of a gesture registration screen;

FIG. 13 is a diagram illustrating a display example of a display screen;

FIG. 14 is a diagram illustrating a display example of a display screen; and

FIG. 15 is a diagram illustrating a display example of a display screen.

### DETAILED DESCRIPTION OF EMBODIMENTS

Hereinafter, one or more embodiments of the present invention will be described with reference to the drawings. While various technically preferable limitations are included in the embodiments described below in order to implement the present invention, the technical scope of the present invention is not to be limited to the following embodiments or illustrated examples.

First, a configuration will be described.

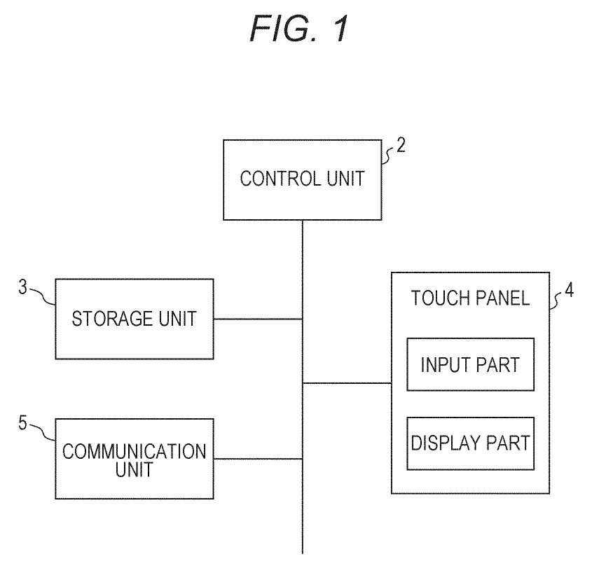

FIG. 1 illustrates an overall configuration of a medical image display apparatus 1 according to the present embodiment.

The medical image display apparatus 1 is a mobile terminal device equipped with a touch panel, including a smartphone and a tablet computer, and is communicably connected to an external device via a network. The medical image display apparatus 1 implemented as a mobile terminal device is also a small terminal suitable for mobile application. The medical image display apparatus 1 in the present embodiment is implemented by adopting a tablet computer.

Note that a network refers to an information communication network constituted with the Internet, a wide area network (WAN), a local area network (LAN), a dedicated line, or a combination of these.

As illustrated in FIG. 1, the above-described medical image display apparatus 1 includes a control unit 2, a storage unit 3, a touch panel 4, and a communication unit 5.

The control unit 2 includes a CPU, a RAM, or the like. The CPU of the control unit 2 reads various programs such as system programs and processing programs stored in the storage unit 3, develops the read programs onto the RAM, and executes various types of processing in accordance with the developed programs.

The storage unit 3 includes a semiconductor nonvolatile memory, or the like. The storage unit 3 stores system programs and various programs necessary for operating the medical image display apparatus 1 in the present embodiment.

The touch panel 4 has a combined function of an input part and a display part, and detects a position that has received touch operation performed by a finger of a user being a doctor, a radiological technician, or the like, an operation member such as a stylus, and then inputs the coordinate data of the detected position. While the present embodiment adopts a capacitance system or a resistive system, other systems may also be adopted.

After detecting touch operation (screen contact operation) on the touch panel 4, the control unit 2 discriminates the touch operation (type) into: touch operation indicating a position within the display screen (terminal screen) of the touch panel 4; the touch operation instructing a change of display content displayed on the display screen (terminal screen); or the touch operation instructing an input of a command or data.

The communication unit 5 includes an interface for data transmission/reception between an external device through wireless communication such as Bluetooth (registered trademark) or Wi-Fi (registered trademark), for example, so as to enable transmission/reception of various types of data including medical image data and various commands concerning related examinations to be described below. Furthermore, the medical image display apparatus 1 can access not merely a specific external device but also other external servers, sites, or the like, through the communication unit 5.

Although not illustrated, the medical image display apparatus 1 appropriately includes a power supply unit, an external connection unit (wired interface), an audio input part, an audio output part, etc. in addition to the above-described control unit 2 or the like.

Furthermore, internal devices such as the control unit 2 and the storage unit 3 are housed inside the touch panel 4 and the exterior casing 6 holding the touch panel 4. An operation unit (not illustrated) such as a button for enabling the internal device to function is assumed to be provided in the exterior casing 6 as necessary.

The medical image display apparatus 1 as described above has a configuration that enables displaying a medical image received from an external device on the display screen of the touch panel 4 on the basis of the control unit 2 and the program stored in the storage unit 3. The medical image display apparatus 1 is communicably connected with an external device storing medical images to perform transmission/reception of various data, signals, or the like including the medical images with the external device so as to construct a medical image display system. In addition, it is possible to change the display mode of a medical image displayed on the display screen on the basis of the operation with a finger, a stylus, or the like.

In the present embodiment, the medical image received from the external device is displayed on the display screen of the touch panel 4. The present invention, however, is not limited to this, and a medical image preliminarily stored in the storage unit 3 may be displayed on the display screen of the touch panel 4.

Although not illustrated, the external device is an intra-hospital server for managing patient information in a hospital, a clinic, or the like. The user usually uses an intra-hospital client terminal, and manages and utilizes patient information while viewing an intra-hospital monitor having a large display screen. The medical image display apparatus 1 can access from outside the hospital to an external device being an intra-hospital server, so as to be able to manage and utilize necessary information.

Next, the configuration concerning the display screen of the touch panel 4 will be described.

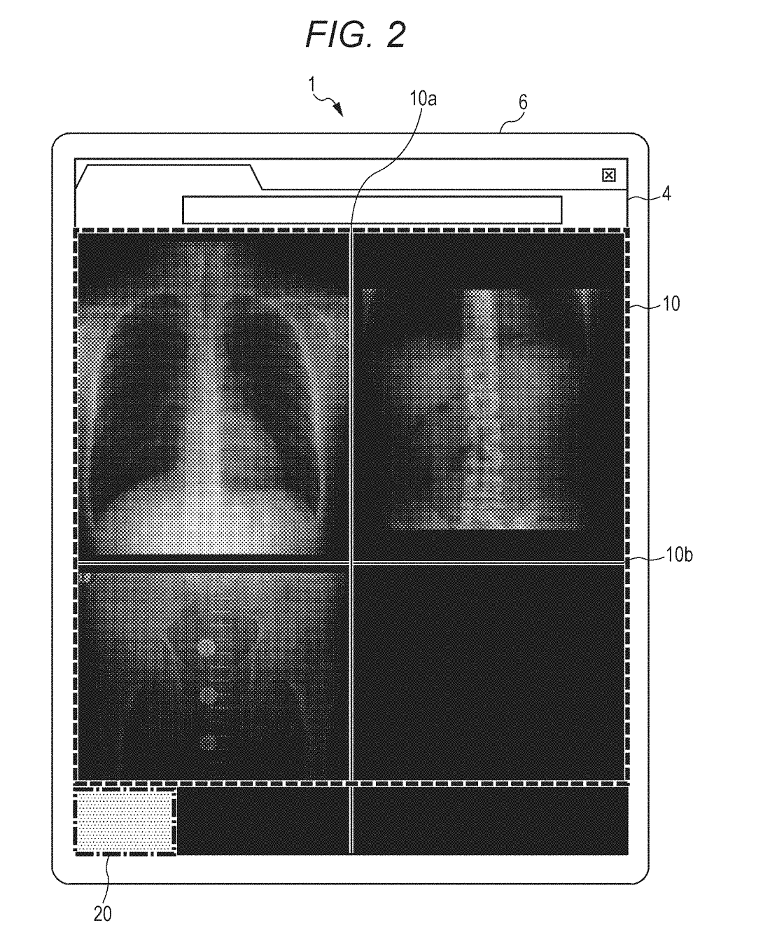

As illustrated in FIG. 2 to FIG. 10, the display screen of the touch panel 4 in the medical image display apparatus 1 includes: an image display region 10 that displays a medical image;

and a small region 20 arranged at a place where the medical image is not displayed.

The display mode of the image display region 10 is set so as to change on the basis of operation by a finger or the like in the small region 20.

The image display region 10 is a region occupying a largest area on the display screen and displays a medical image received from the intra-hospital server as an external device.

The image display region 10 according to the present embodiment is partitioned into four regions by partition lines 10a and 10b so as to be able to display mutually different medical images in each of these four regions.

Note that the partition lines 10a and 10b may be configured to be movable in the image display region 10 or switchable between a display state and a non-display state. Furthermore, it is also possible to change the manner of partitioning the image display region 10 by increasing or decreasing the number of the partition lines 10a and 10b. This makes is possible to flexibly change the layout in the image display region 10 by the user.

The change in the display mode of the image display region 10 includes, for example, a change in a manner of dividing the image display region 10, a pan display of the medical image displayed in the image display region 10 (a viewpoint shift in the vertical and horizontal directions), a zoom display of the medical image (enlargement/reduction), adjustment of contrast, gradation, color tone, resolution, etc. of a medical image, switching to other screens such as a gesture registration screen, display/non-display of related examination list, and display/non-display of a tool box. Such a change in the display mode can be performed by operation with a finger or the like in the small region 20.

Note that since the image display region 10 is provided on the touch panel 4, the display mode may be changed by performing operation specific to the touch panel 4 without using the small region 20. For example, one of medical images displayed in the image display region 10 can be enlarged by performing pinch-out operation, or conversely, can be reduced by performing pinch-in operation, within the segmented region. In addition to this, it is also possible to temporarily exclude from the image display region 10 by flick operation or swipe operation, or download a medical image to the storage unit 3 of the medical image display apparatus 1 by performing long tap operation.

In this case, however, since the operation is performed on the medical image displayed in the image display region 10, the medical image might be hidden by a finger or a hand. Therefore, the change in the display mode based on such operation is merely operation specific to the touch panel 4, and would not attach any limitation to the present invention. Therefore, operation related to the change in the display mode in the image display region 10 performed during the display of the small region 20 in the present embodiment may also be defined to be based solely on the operation with a finger or the like in the small region 20.

As illustrated in FIG. 2 or the like, the small region 20 occupies a smaller area on the display screen, as compared with the area occupied by the image display region 10. In addition, although the small region 20 is formed in a rectangular shape in the present embodiment and has a different color from the background color of the display screen (the image display region 10), there is no limitation in the shape or color, and other shapes and colors may be used.

Still, by using the color of the small region 20 different from the background color of the display screen (image display region 10), it would be advantageous in that the user can easily confirm the location of the small region 20. In other words, it would be sufficient as long as the user can confirm the location of the small region 20, and thus, the same effect can be obtained by bordering the small region 20, for example.

The small region 20 is arranged at a position along an edge portion of the display screen of the touch panel 4. More specifically, the small region 20 in the present embodiment is arranged at one corner on the lower side of four corners of the display screen. In other words, the small region 20 is arranged at a position along the lower edge portion of the display screen.

Note that the position of the small region 20 on the display screen is preferably at least within a reach of a thumb when the medical image display apparatus 1 having the exterior casing 6 is held by one hand. Moreover, even when there is a small region 20 within a reach of the thumb, it would be still difficult to operate if the thumb just reaches the edge of the small region 20. That is, the small region 20 also needs to have enough area to enable operation with a thumb. In short, it is assumed that the small region 20 is set to a position/area enabling operation with a thumb when the medical image display apparatus 1 is held with one hand. Note that, since the size of the hand and the length of the finger are different person by person, it would be just a rough guide.

Accordingly, the position of the small region 20 may be set changeable. That is, it is sufficient as long as the small region 20 is set to a position/area enabling operation with a thumb when the medical image display apparatus 1 is held with one hand, and thus, may be configured to be changeable to a certain position under that condition. The position change may be performed automatically or may be performed manually. In the case of manual operation, for example, long tap operation is performed on the small region 20 to cancel the fixation of the small region 20 on the display screen to move the small region 20 to a desired position without releasing the finger or the like as it is, and then, moving operation of releasing the finger or the like is performed to fix the position again.

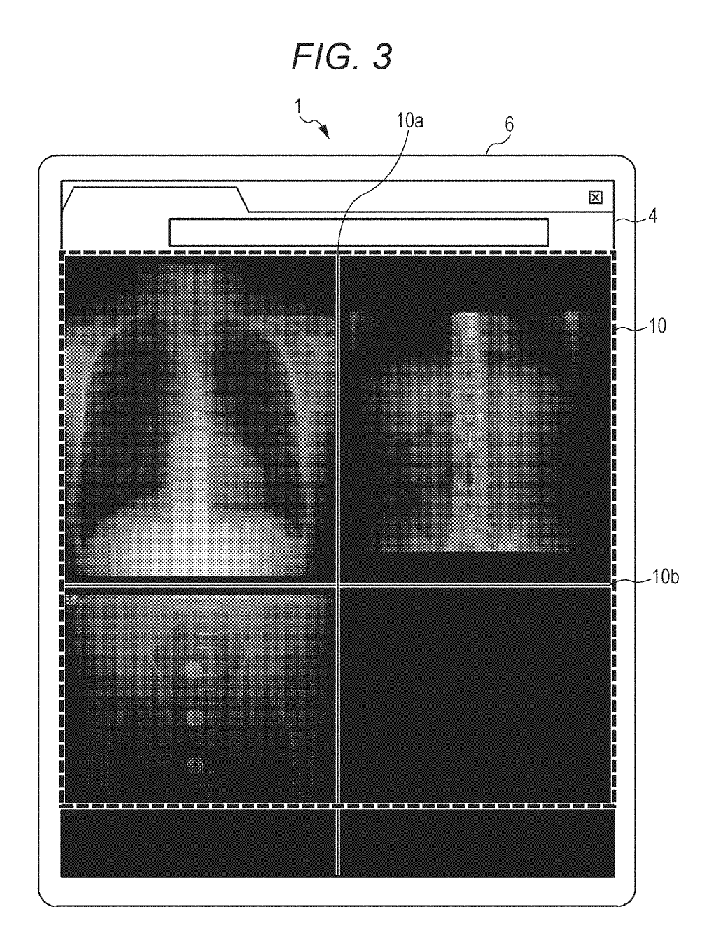

Furthermore, when the non-operated state continues for a predetermined time or longer, or when a position the outside of the small region 20 is touched in the display screen, the small region 20 turns into a non-display state as illustrated in FIG. 3. Turning the small region 20 to the non-display state in this manner enables suppression of hindrance of visual confirmation of a medical image displayed in the image display region 10 due to the existence of the small region 20, making it possible to prevent degradation of visibility of the medical image.

Subsequently, a case where the display mode of the image display region 10 is changed on the basis of the operation by the finger or the like in the small region 20 will be described more specifically.

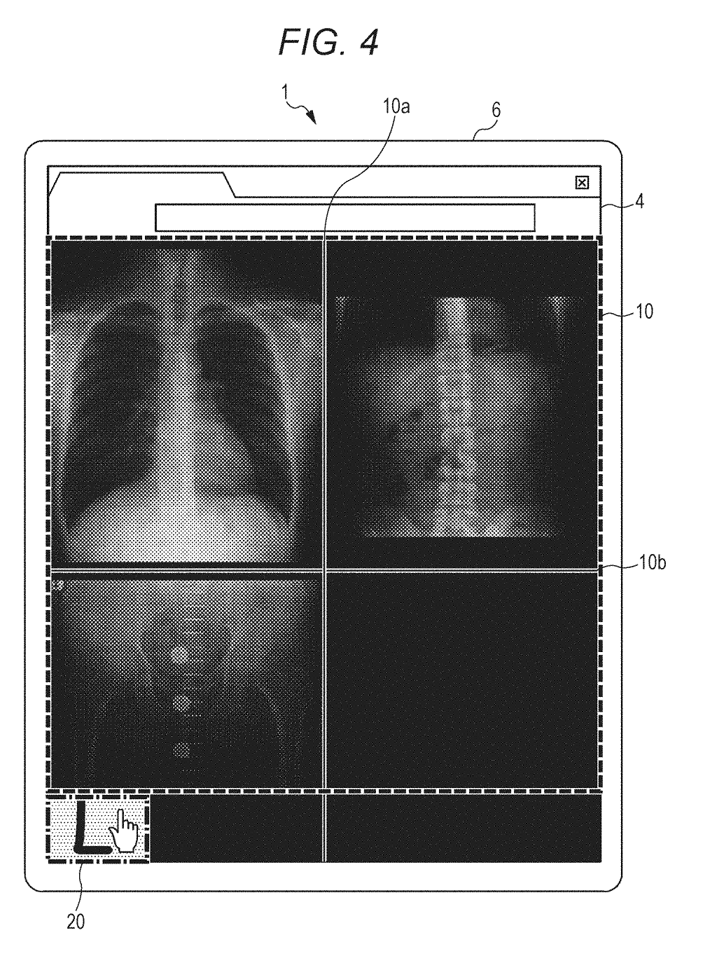

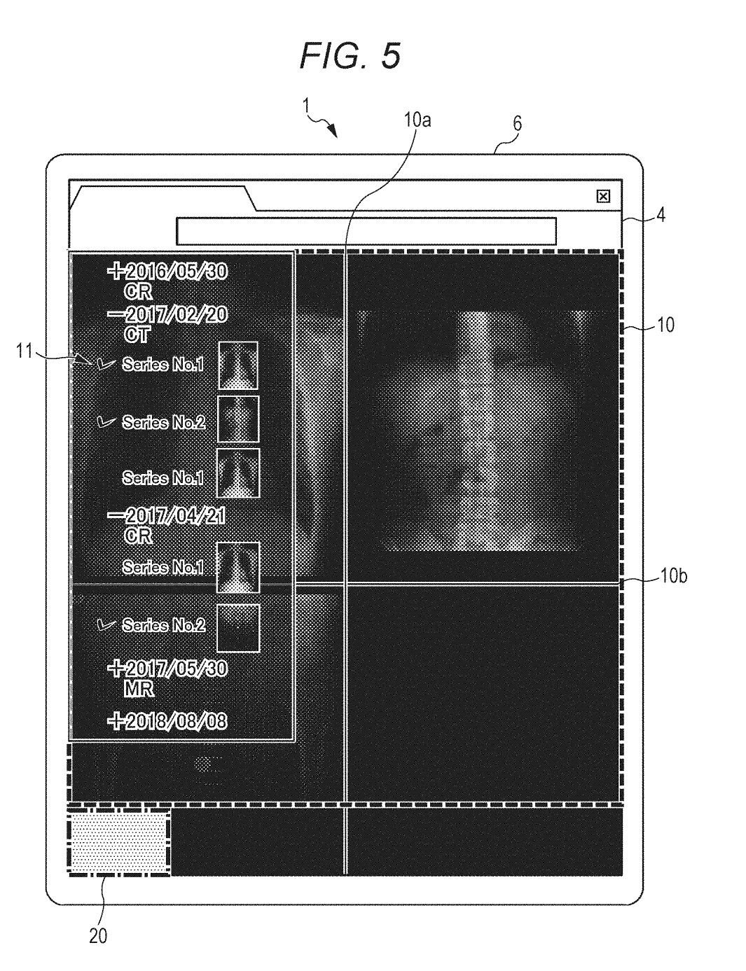

In the present embodiment, for example, as illustrated in FIG. 4, by inputting “L” in the small region 20 with a finger, a stylus, or the like, it is possible to display a related examination list 11 in the image display region 10 as illustrated in FIG. 5.

Hereinafter, in the present embodiment, letters, signs, marks, symbols, or the like, that are input in the small region 20 by a finger, a stylus, or the like will be referred to as gestures. Additionally, operation itself performed the finger, the stylus or the like in the small region 20 may also be referred to as a gesture.

The related examination refers to all related examination performed on a certain patient and all related examination performed at the same site of the body of a patient, and medical images related to such examination are stored in the storage unit of an intra-hospital server being an external device.

Furthermore, the medical image concerning the related examination is retrieved with reference to supplementary information of each of the images. For example, in a case where an image has supplementary information having the same patient name, the patient ID, the examination site and different examination date, namely, before or after the certain date, the medical image can be discriminated as a medical image of the related examination.

The related examination list 11 is a list of details of the related examination (date/modality type) as described above.

Note that the modality refers to the type of the imaging apparatus that photographs the medical image displayed in the image display region 10. Examples of the type of the imaging apparatus include a radiography apparatus (Flat Panel Detector), a breast imaging apparatus (mammography), Computed Tomography (CT), Magnetic Resonance Imaging (MRI), and Computed radiography (CR).

Tap operation performed on each of items in the related examination list 11 would unfold a thumbnail list, and re-tap operation on the item makes it possible to fold the thumbnail list to non-display. Further, tap operation on the thumbnail makes it possible to display a medical image corresponding to the thumbnail, in the image display region 10.

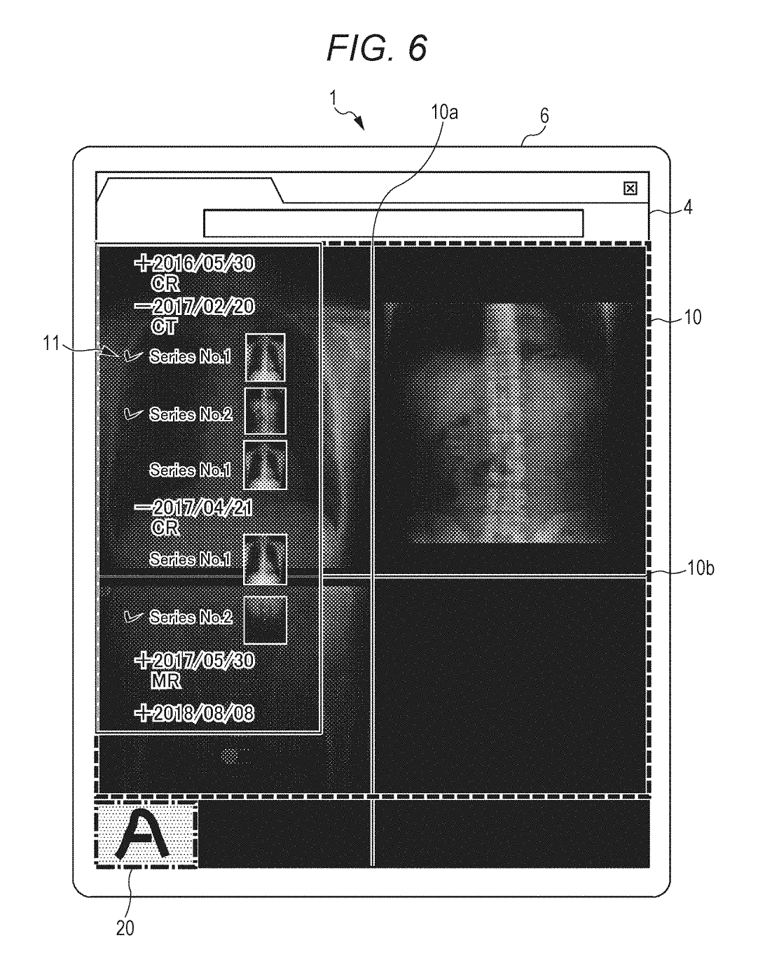

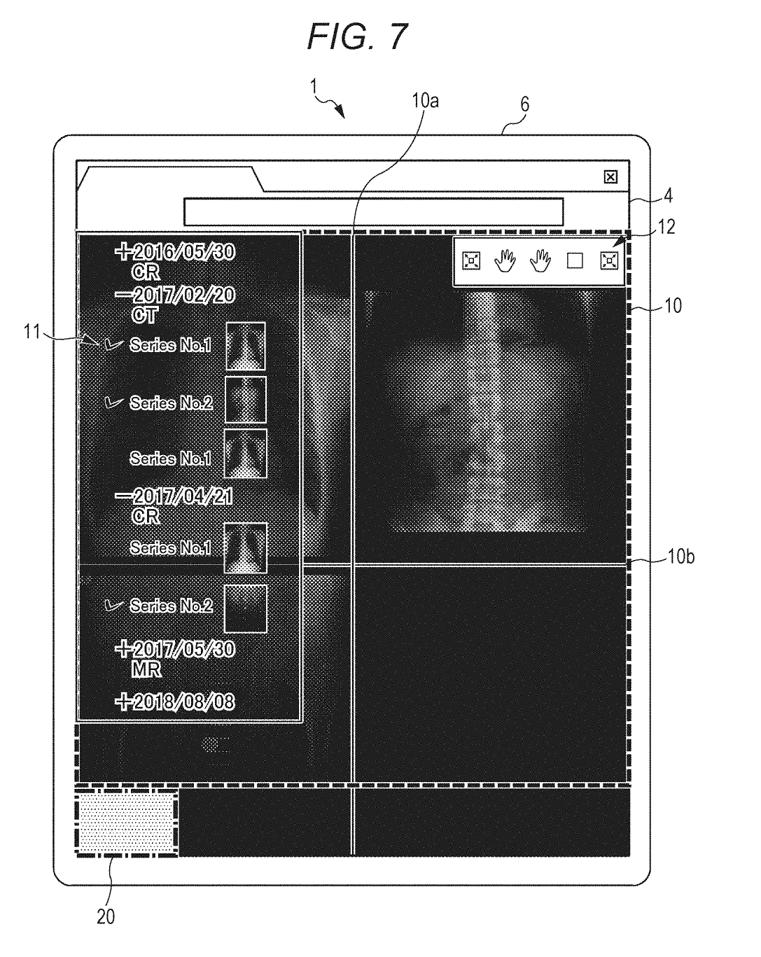

Furthermore, for example, as illustrated in FIG. 6, by inputting a gesture of “A” in the small region 20 with a finger, a stylus, or the like, it is possible to display a tool box 12 in the image display region 10 as illustrated in FIG. 7.

The tool box 12 includes a plurality of function buttons. These function buttons include various types of buttons, for example, a button for switching display/non-display of supplementary information of each of medical images in the image display region 10. Various buttons can be activated by a tap operation.

While inputting a gesture in the small region 20 enables display of the related examination list 11 and the tool box 12 in the image display region 10 as described above, re-inputting the same gesture would make them non-display.

Furthermore, it is assumed that the input of the gesture can be continuously performed within a range that can be recognized and discriminated by the control unit 2.

Gestures can be preliminarily stored in the storage unit 3 of the medical image display apparatus 1. That is, the gestures can be preliminarily registered.

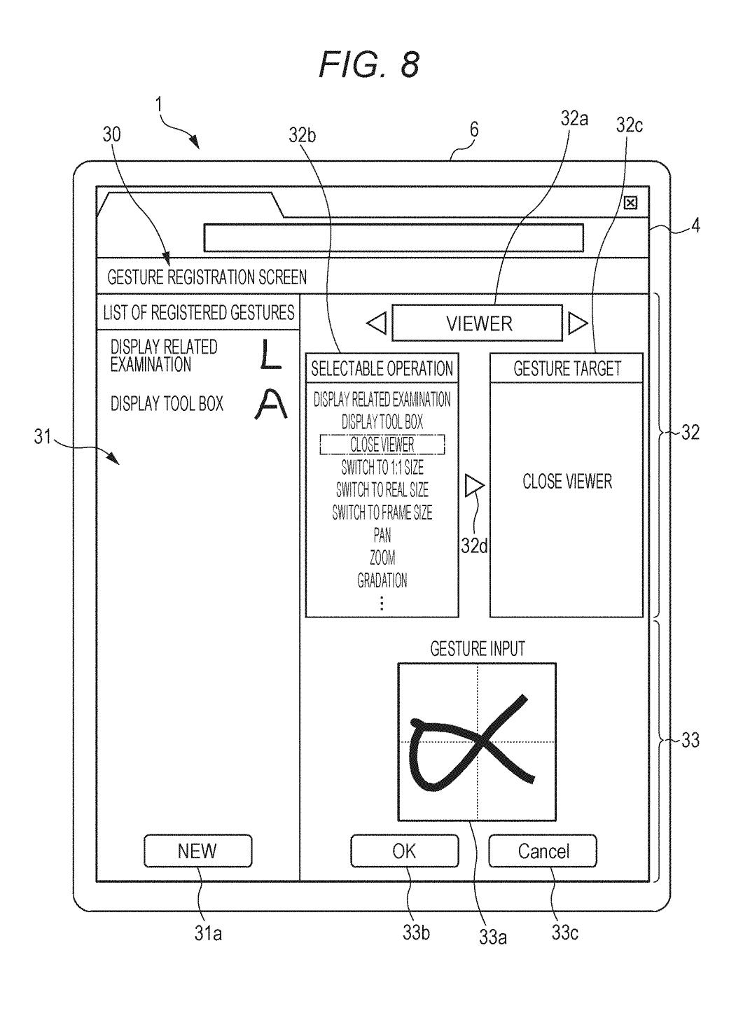

Inputting a predetermined gesture in the small region 20 would enable display of a gesture registration screen 30 on the display screen of the touch panel 4 as illustrated in FIG. 8.

Registration of gestures can be performed on the screen 30.

The gesture registration screen 30 includes a registered gesture list 31, an operation selection field 32, and a gesture input field 33.

The registered gesture list table 31 displays registered gestures. In addition, a new creation button 31a is provided.

In response to tap operation on the new creation button 31a, the operation selection field 32 is activated to make it possible to select operation to be associated with a new gesture.

The operation selection field 32 includes a classification selection part 32a for selecting classification of selectable operation, a selectable operation list 32b including a list of selectable operation and a gesture target field 32c that displays operation selected by the user from the selectable operation list 32b, and a selection button 32d.

The user first selects the classification of the selectable operation from the classification selection part 32a. In the example illustrated in FIG. 8, “Viewer” is selected.

The classification “Viewer” includes display of various selectable operation items as illustrated in the drawing. The user can either use drag operation from among them or tap operation on the selection button 32d after operation selection to select selectable operation.

When the operation selected by the user is displayed in the gesture target field 32c, the gesture input field 33 is activated and enables setting of an arbitrary gesture for the selected operation.

The gesture input field 33 includes an input part 33a for inputting a gesture, an OK button 33b, and a cancel button 33c.

The control unit 2 can recognize the gesture input to the input part 33a by the user and can check the presence or absence of the same gesture by collating with the already registered gesture. In a case where the same gesture is not registered, registration of the gesture is completed by tap operation on the OK button 33b. The input of the gesture can be redone by tap operation on the cancel button 33c. In a case where the same gesture has already been registered, registration is disabled. In such a case, for example, it is allowable to set such that the gesture input to the input part 33a is to be erased by tap operation on the OK button 33b.

In the example illustrated in FIG. 8, the operation “Close the viewer” is selected, and the gesture of “a” has been input.

Here, “viewer” refers to software enabling display including the image display region 10 and the small region 20, and is also referred to as a browser.

Specifically, when the user inputs “a” in the small region 20, the display including the image display region 10 and the small region 20 is closed and another screen (for example, a home screen of the terminal) is to be displayed on the display screen of the touch panel 4.

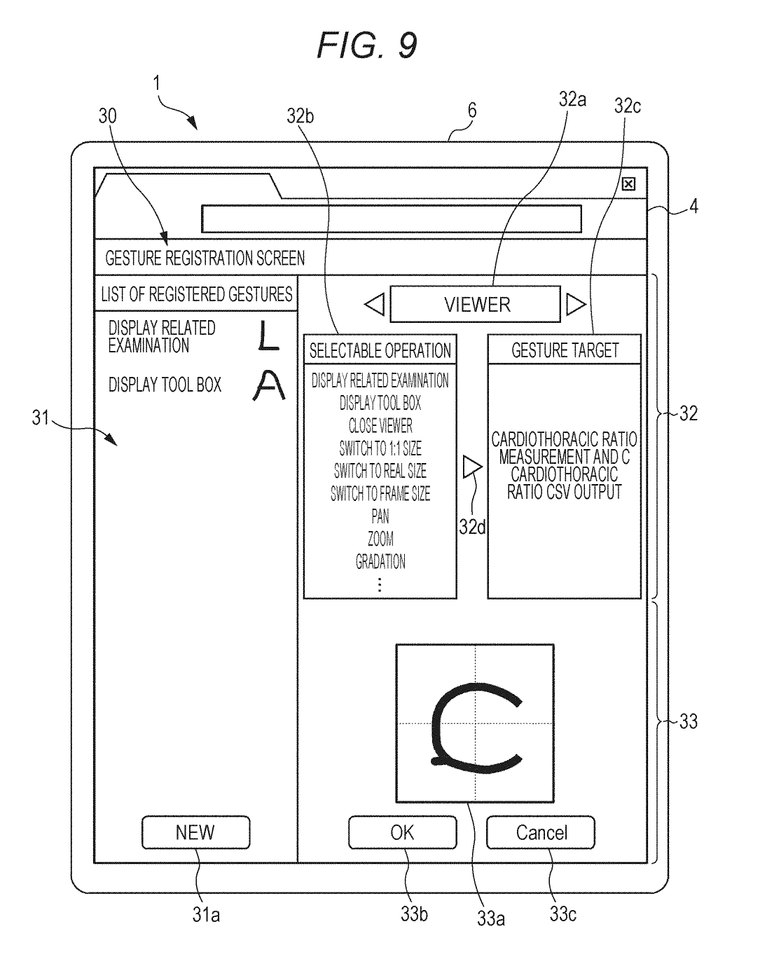

While the above has described the gesture registration corresponding to single operation, the medical image display apparatus 1 in the present embodiment also can perform gesture registration corresponding to a plurality of types of operation as illustrated in FIG. 9. In other words, the display mode of the image display region 10 can be set as a workflow in which changes are made in a plurality of stages. That is, by merely inputting a single gesture in the small region 20, it is possible to sequentially perform a plurality of changes in the display mode in the image display region 10.

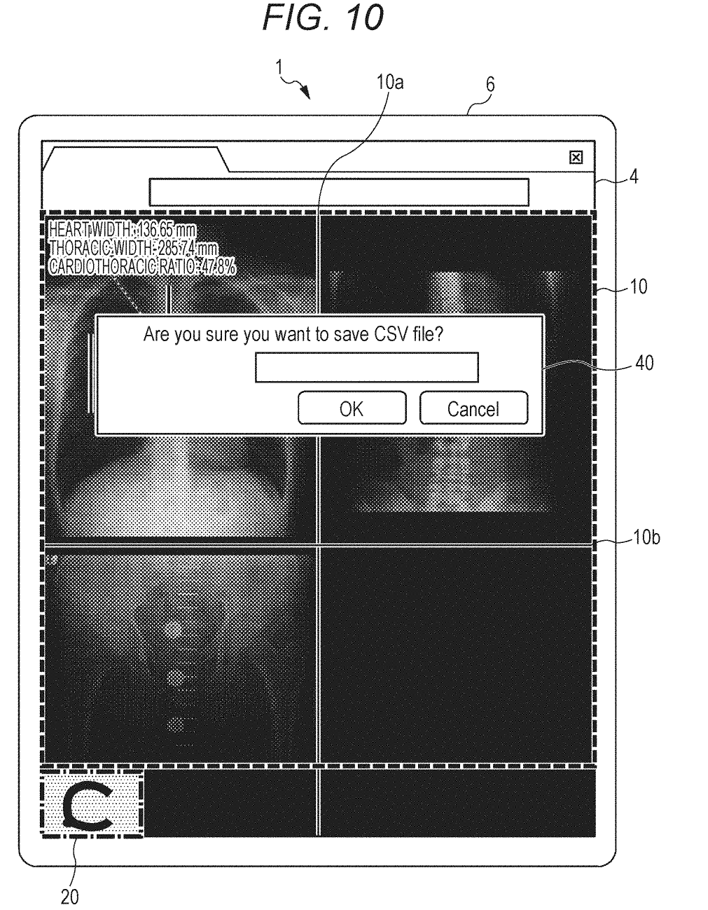

More specifically, an example illustrated in FIG. 9 is an example in which operation of “automatic cardio thoracic ratio measurement” and operation of “C cardiothoracic ratio CSV output” are selected by the user, and the gesture of “C” has been input. When the registration is completed, by inputting “C” in the small region 20 as illustrated in FIG. 10, the cardio thoracic ratio in the chest front X-ray image is measured in a displayed medical image, and then, it is possible to display, in the image display region 10, a window 40 asking the user to select whether or not to save a comma-separated values (CSV) file including records of the result.

The medical image display apparatus 1 configured as described above is not particularly limited in use. Still, it is assumed that the medical image display apparatus 1 be used in a case where a user such as a doctor is located away from the intra-hospital client terminal and a medical image needs to be confirmed.

The medical image display apparatus 1 in the present embodiment is implemented by a tablet type computer. However, it is not limited thereto, and a smaller terminal such as a smartphone may be adopted. In a case where a medical image display apparatus smaller than a tablet computer is adopted, using similar layouts of the display screen might make the medical image too small or make it difficult to perform input operation within the small region 20. Therefore, in a case where a compact terminal is adopted as a medical image display apparatus, it is desirable to make the display screen layout suitable for a small medical image display apparatus. For example, the image display region 10 may have a single screen layout without the partition lines 10a and 10b.

Furthermore, various functions in the medical image display apparatus 1 as described above may be provided as applications.

As described above, according to the medical image display system of the present embodiment, there is provided the medical image display apparatus 1 that displays a medical image on a display screen of the touch panel 4, and the display mode of the medical image displayed on the display screen can be changed on the basis of the screen contact operation onto the display screen, and the display screen includes the image display region 10 that displays a medical image and a small region 20 being a region smaller than the image display region 10 and arranged at a position where the medical image is not displayed, and the display mode of the image display region 10 is set so as to change on the basis of the screen contact operation in the small region 20, making it possible to reduce the portion at which operation for changing the display mode of the image display region 10 is performed. With this configuration, when performing operation for changing the display mode of the image display region 10, the motion would not disturb viewing of the image display region 10, facilitating viewing of the medical image displayed in the image display region 10. Furthermore, when the portion to be operated is small, operation with one hand would be also possible. This leads to suppression of the degradation of visibility of the medical image displayed on the display screen and enhancement of operability.

Furthermore, since the small region 20 is arranged at a position along the edge portion of the display screen of the touch panel 4, it is possible to achieve easy operation when the medical image display apparatus 1 is held with one hand, and unlikely to hinder operation of the image display region 10. Therefore, this can suppress the degradation of visibility of the medical image displayed on the display screen and can enhancement operability.

In addition, since the small region 20 is arranged at a position along the lower edge portion of the display screen of the touch panel 4, it is possible to operate the medical image display apparatus 1 by hand without hiding the image display region 10 as much as possible. Therefore, this can suppress the degradation of visibility of the medical image displayed on the display screen and can enhancement operability.

Moreover, operation (gesture) using a finger or the like in the small region 20 is preliminarily registered, and a change in the display mode in the image display region 10 (the related examination list 11, the display/workflow of the tool box 12) has been set corresponding to the preliminarily registered operation (gesture) by a finger or the like in the small region 20. Accordingly, by just performing preliminarily registered operation using a finger or the like in the small region 20, it is possible to make a change in the display mode corresponding to the operation, in the image display region 10.

In addition, the display mode of the image display region 10 can be set as a workflow in which changes are made in a plurality of stages. Therefore, by just performing single operation by a finger or the like in the small region 20, it is possible to make a change in display mode in the image display region 10.

Furthermore, according to the medical image display apparatus 1 of the present embodiment, it is possible to change the display mode of a medical image displayed on a display screen on the basis of screen contact operation on the display screen of the touch panel 4, and the display screen includes the image display region 10 that displays a medical image and a small region 20 being a region smaller than the image display region 10 and arranged at a position where the medical image is not displayed, and the display mode of the image display region 10 is set so as to change on the basis of the screen contact operation in the small region 20, making it possible to reduce the portion at which operation for changing the display mode of the image display region 10 is performed. With this configuration, when performing operation for changing the display mode of the image display region 10, the motion would not disturb viewing of the image display region 10, facilitating viewing of the medical image displayed in the image display region 10. Furthermore, when the portion to be operated is small, operation with one hand would be also possible. This leads to suppression of the degradation of visibility of the medical image displayed on the display screen and enhancement of operability.

### Configuration Example

The embodiments to which the present invention is applicable are not limited to the above-described embodiments, and can be appropriately changed without departing from the scope and spirit of the present invention. Hereinafter, a configuration example will be described. The following configuration examples may be combined whenever possible.

### Configuration Example 1

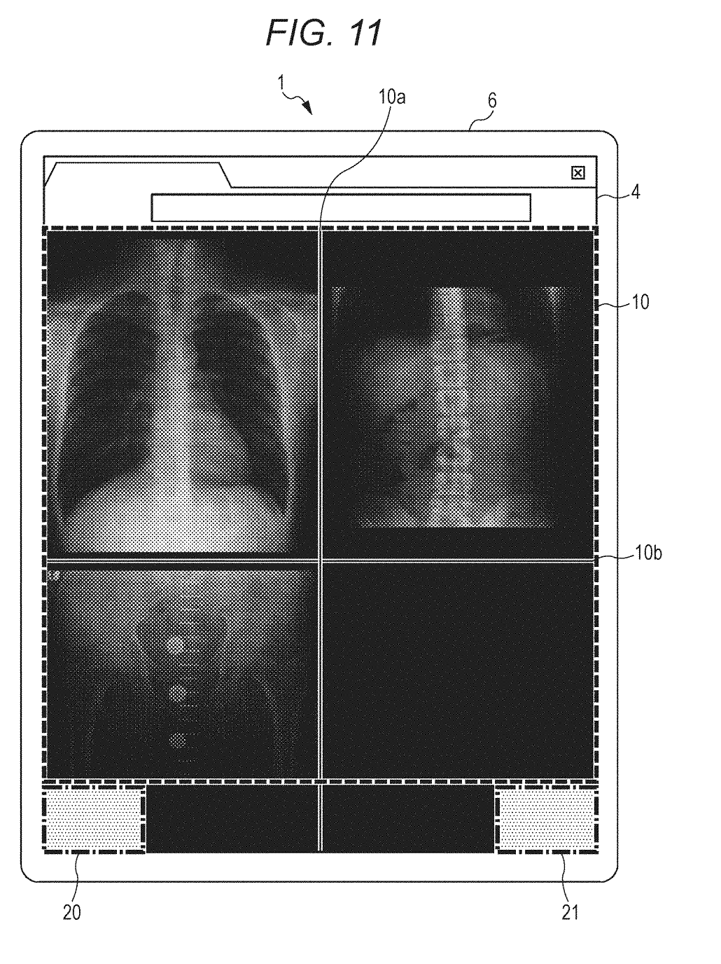

As illustrated in FIG. 11 to FIG. 14, a plurality of small regions 20 and 21 in the present configuration example is arranged on the display screen of the touch panel 4.

While the present configuration example uses a configuration in which the two small regions 20 and 21 are arranged on the display screen, three or more small regions may be provided, and the number is not particularly limited.

The plurality of small regions 20 and 21 is arranged at positions along the edge portions of the display screen of the touch panel 4. More specifically, one small region 20 in the present configuration example is arranged at one corner on the lower side of the four corners of the display screen, while the other small region 21 is arranged at the other corner on the lower side of the four corners of the display screen.

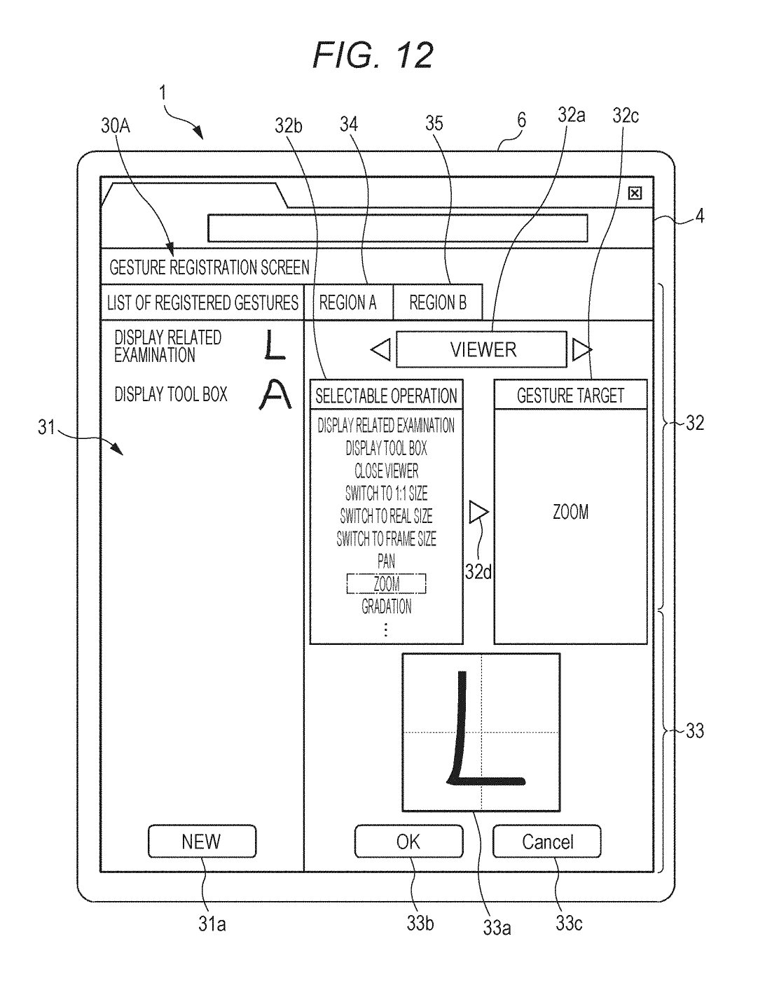

Gestures onto such small regions 20 and 21 can be registered from a gesture registration screen 30A as illustrated in FIG. 12. Similarly to the above-described embodiment, the gesture registration screen 30A includes the registered gesture list 31, the operation selection field 32, and the gesture input field 33, so as to function similarly to the case of the embodiment. The gesture registration screen 30A includes small region selection parts 34 and 35.

The small region selection part 34 is a tab selected by tap operation when a gesture to be input to one small region 20 is registered.

The small region selection part 35 is a tab selected by tap operation when a gesture to be input to the other small region 21 is registered.

Registration of a gesture to be input to one of the small regions 20 is similar to the above embodiment, and the gesture to be input to the other small region 21 is registered after execution of tap operation on the small region selection part 35.

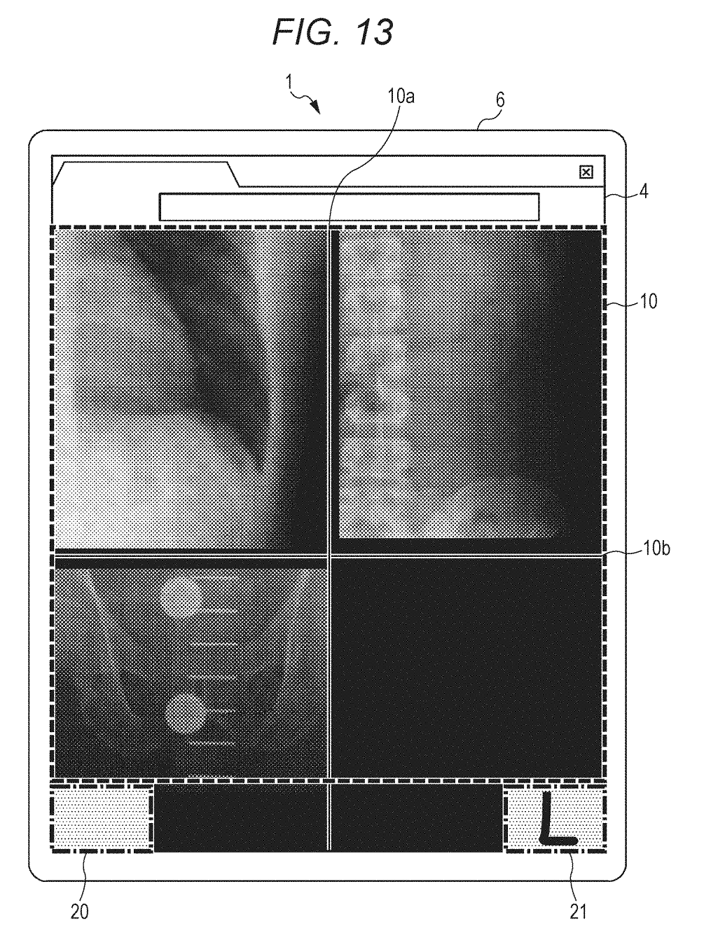

In the example illustrated in FIG. 12, “L” is registered as a gesture input to the other small region 21. “Zoom (enlarge)” is selected as a change in the display mode in the image display region 10. That is, as illustrated in FIG. 13, by inputting “L” to the other small region 21, it is possible to zoom (enlarge) a medical image displayed in the image display region 10.

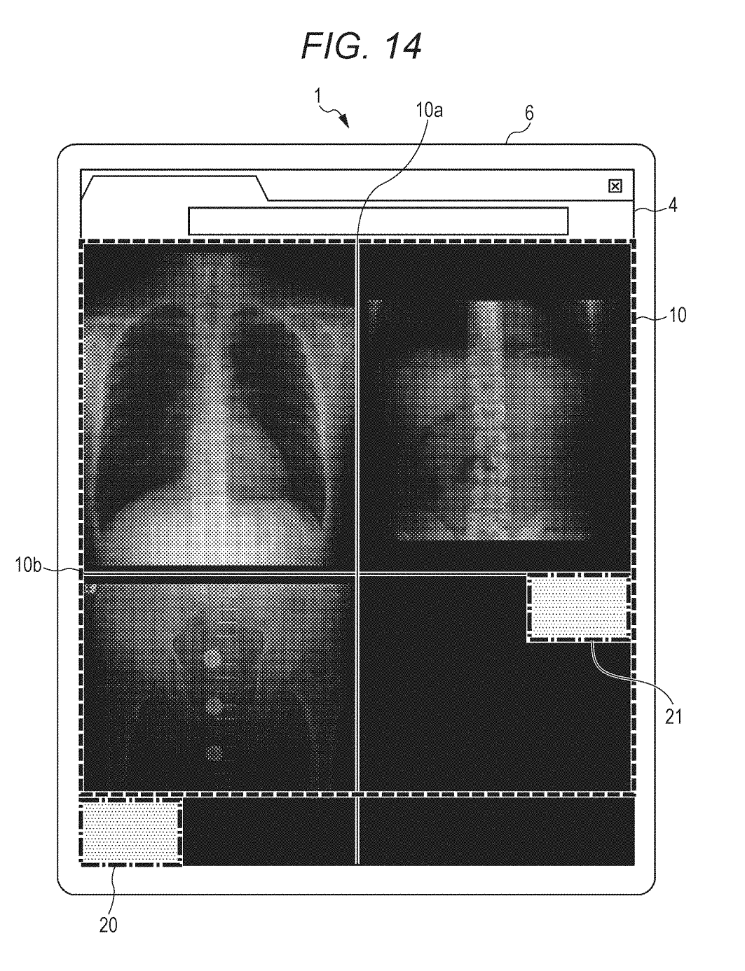

Note that although one small region 20 in the present configuration example is arranged at one corner on the lower side of the four corners of the display screen, while the other small region 21 is arranged at the other corner on the lower side of the four corners of the display screen, the present invention is not limited to this pattern. That is, the small regions 20 and 21 are arranged at positions along the edge portion of the display screen of the touch panel 4, as illustrated in FIG. 14, and thus, it is allowable to arrange the small regions 20 and 21 on a central side of the edge portion of the display screen, instead of at the four corners of the display screen.

The example illustrated in FIG. 14 is a case where the small region 21 enters a portion of the image display region 10 in which no medical image is displayed. However, in a case where a medical image is displayed, the small region 21 is assumed to be automatically arranged at a vacant corner on the display screen

According to such a configuration, the plurality of small regions 20 and 21 is arranged on the display screen of the touch panel 4, making it possible to prevent complication of the operation in the small regions 20 and 21. For example, if the number of small regions is one and gesture registration is continued one after another, gesture would be complicated gradually in some cases. Complication of gesture in this manner might cause a situation in which the control unit 2 has difficulty in distinguishing the gesture or the situation in which the user cannot remember the gestures. In view of this, the present configuration example arranges a plurality of small regions 20 and 21 on the display screen of the touch panel 4, and this enables making changes in different display modes in the image display region 10 with the same gesture input. With this configuration, it is possible to increase the number of selectable operation items without increasing the number of gestures.

### Configuration Example 2

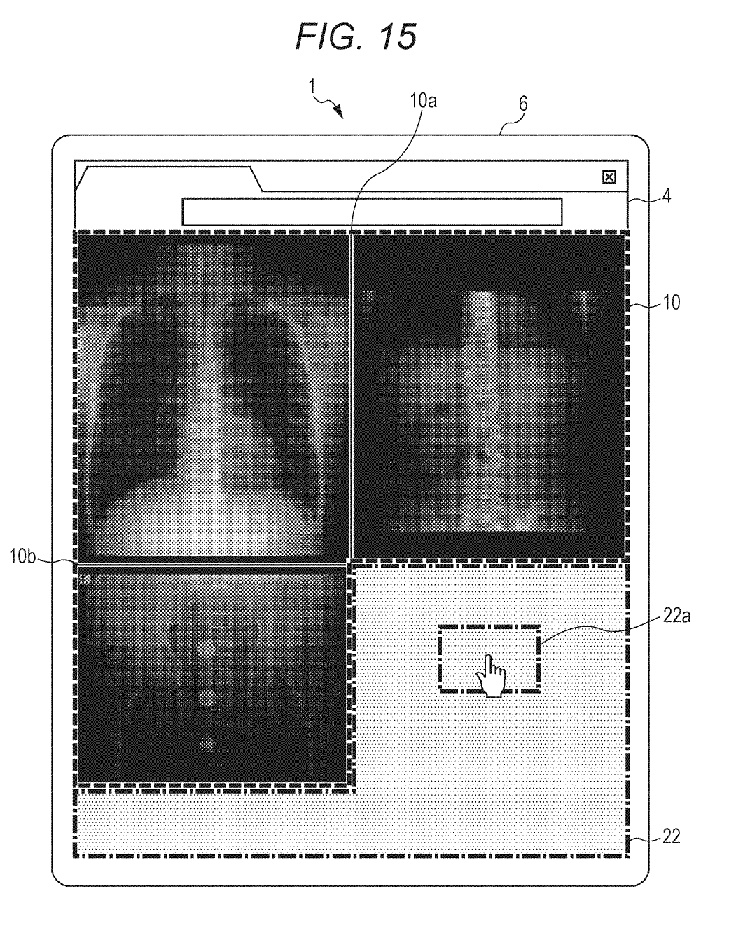

As illustrated in FIG. 15, a small region 22 in the present configuration example corresponds to a whole region in which no medical image is displayed, among the display screen of the touch panel 4. Within this small region 22, a range of an input region 22a can be defined with a portion touched by the user as a starting point. In other words, in the present configuration example, portions around the position touched by the user are set to be the input region 22a into which gestures can be input, among the portions (small regions 22) where no medical image is displayed on the display screen.

With this configuration, it is possible to change the position of the input region 22a in which the gesture can be input in accordance with a medical image displayed in the image display region 10, making it possible to enhance convenience and operability.

Although embodiments of the present invention have been described and illustrated in detail, the disclosed embodiments are made for purposes of illustration and example only and not limitation. The scope of the present invention should be interpreted by terms of the appended claims.

What is claimed is:

1. A medical image display system comprising
a medical image display apparatus that displays a medical image on a display screen of a touch panel,
the medical image display system being capable of changing a display mode of the medical image displayed on the display screen on the basis of screen contact operation on the display screen,
wherein the display screen includes:
an image display region that displays the medical image; and
a small region being a region smaller than the image display region and arranged at a position where the medical image is not displayed, and
a display mode of the image display region is set so as to change on the basis of the screen contact operation in the small region.

2. The medical image display system according to claim 1,
wherein the small region is arranged at a position along an edge portion of the display screen.

3. The medical image display system according to claim 2,
wherein the small region is arranged at a position along a lower edge portion of the display screen.

4. The medical image display system according to claim 1,
wherein a plurality of the small regions is arranged on the display screen.

5. The medical image display system according to claim 1,
wherein operation by a finger or the like in the small region is preliminarily registered, and
the change in the display mode in the image display region is set corresponding to the operation by the finger or the like in the preliminarily registered small region.

6. The medical image display system according to claim 1,
wherein the display mode of the image display region can be set as a workflow in which changes are made in a plurality of stages.

7. A medical image display apparatus capable of changing a display mode of a medical image displayed on a display screen of a touch panel on the basis of screen contact operation on the display screen of the touch panel,
wherein the display screen includes:
an image display region that displays the medical image; and
a small region being a region smaller than the image display region and arranged at a position where the medical image is not displayed, and
a display mode of the image display region is set so as to change on the basis of the screen contact operation in the small region.
